(19) United States
(12) Patent Application Publication
(10) Pub. No.: US 2015/0320965 A1
(43) Pub. Date: Nov. 12, 2015

(54) SCENT SYSTEM FOR AMBIENT EXPERIENCE

(71) Applicant: KONINKLIJKE PHILIPS N.V., EINDHOVEN (NL)

(72) Inventors: WILHELMUS DANIEL HYACINTHUS VAN GRONINGEN, EINDHOVEN (NL); MURRAY FULTON GILLIES, EINDHOVEN (NL); PETER ARNOLD MARIA VAN KOLLENBURG, BEST (NL)

(21) Appl. No.: 14/405,172

(22) PCT Filed: Jun. 4, 2013
(86) PCT No.: PCT/IB2013/054583

§ 371 Date: Dec. 3, 2014

Related U.S. Application Data
(60) Provisional application No. 61/656,565, filed on Jun. 7, 2012.

(51) Int. Cl. A61M 21/00

(52) U.S. Cl. CPC A61M 21/00; A61M 2021/0016; A61M 2021/0027; A61M 2230/63; A61M 2205/502

(57) ABSTRACT

The invention regards a system (102, 502, 702, 802) for generating an ambient healing environment in a room (101, 701, 801) for performing a medical procedure. For that purpose the system comprises a primary scent supply mechanism (104, 508, 708, 804) configured for providing a patient (105, 805) with consecutive batches of scent (204, 206, 208, 304, 306, 308, 404, 406, 408). The system furthermore comprises a controller (106, 510, 710, 806) arranged for controlling the primary scent supply mechanism (104, 508, 708, 804) such that it provides during operation the consecutive batches of scent intermittently according to a primary time sequence (202, 302, 402, 602, 902).

### FIELD OF THE INVENTION

The invention relates to a system for generating an ambient healing environment in a room for performing a medical procedure.

### BACKGROUND OF THE INVENTION

U.S. Pat. No. 7823306 B1 discloses a room for conducting medical procedures including devices to induce calmness in a patient during a medical procedure. One such device is a scent device for providing smells. However, without taking appropriate measures, such scent device may have a stress inducing rather than a calming effect due to providing a surplus of scent.

### SUMMARY OF THE INVENTION

It is an object of the invention to provide a system for generating an ambient healing environment in a room, more particularly a room for performing a medical procedure, a waiting room in a hospital or a hospital's dressing room, which system is capable of reducing patient stress using scent.

This object is achieved by the system according to the invention, comprising a primary scent supply mechanism configured for providing a patient with consecutive batches of scent, and a controller arranged for controlling the primary scent supply mechanism such that it provides during operation the consecutive batches of scent intermittently according to a primary time sequence.

The system according to the invention employs the principle that a scent may be provided intermittently without comprising its continuous calming effect. That is, depending on the particular scent, a scent remains in a patient's memory for some period of time. By intermittently providing consecutive batches of scent, i.e. by continuously alternating between zero scent and a non-zero quantity of scent, the probability of a scent surplus is reduced without affecting the potential to reduce patient stress. Herein the period of time between consecutive batches of scent should relate to the period of time that such scent remains in a patient's memory. The larger the size of such consecutive batches of scent, the larger the period in between such batches may be. Depending on the specific type of scent, via experimental verification the person skilled in the art is to determine a size of the consecutive batches of scent as well as a period of time between consecutive batches of scent.

The system according to the invention is not limited to a particular type of scent. Anyhow essential oils or blends thereof, which are known for their relaxing effect, may be successfully employed in conjunction with the system according to the invention.

In a preferred embodiment of the system according to the invention, the system comprises a sensor for generating a measurement signal indicative for an amount of scent in the room, and wherein the controller is configured for adapting in response to the measurement signal a period of time between consecutive batches of scent. This embodiment enables increasing the period of time between consecutive batches in response to the measurement signal indicating an excess of scent in the room for performing the medical procedure as well as decreasing said period if the measurement signal indicates a lack of scent. This embodiment enables tailoring the period of time in between consecutive batches of scent to prevent either a surplus of scent or a shortage of scent in the room for performing the medical procedure. As a result, this embodiment advantageously further reduces a patient's stress.

In a preferred embodiment of the system according to the invention, the system comprises a sensor for generating a measurement signal indicative for an amount of scent in the room, and wherein the controller is configured for adapting in response to the measurement signal a size of consecutive batches of scent. The size of a batch regards the amount of scent per consecutive batch in terms of kg or mol. Such batch size is controllable either by adapting the mass flow rate from the primary supply mechanism or by changing the period during which said mass flow rate is generated by the primary supply mechanism. This embodiment enables decreasing the size of consecutive batches in response to the measurement signal indicating an excess of scent in the room for performing the medical procedure as well as increasing said size if the measurement signal indicates a lack of scent. This embodiment enables tailoring the batch size to prevent from surpluses as well as shortages of scent in the room for performing the medical procedure. As a result, this embodiment has the advantage of further reducing a patient's stress.

In a further preferred embodiment of the system according to the invention, the controller is configured to initiate the primary supply time sequence in response to detecting the patient has entered the room. The controller may detect patient entry for example by an odd numbered instance of a door being opened and closed or via RFID tracking This embodiment enables providing scent when necessary i.e. only if a patient is present in the room thereby preventing from both a surplus and a shortage of scent. Consequently this embodiment advantageously further reduces the patient's stress.

In a further preferred embodiment of the system according to the invention, the controller is configured to terminate the primary supply time sequence in response to detecting the patient has left the room. The controller may detect patient leave for example by RFID tracking or via an even numbered instance of a door being opened and closed. This embodiment enables providing scent when needed i.e. only if the patient is actually present in the room. Consequently this embodiment is advantageous in that it further reduces a patient's stress.

In a further preferred embodiment of the system according to the invention, the controller is configured to initiate extracting scent from the room in response to detecting the patient has left said room. This embodiment effectively prevents from ill-defined intermixing of subsequent scents. Such ill-defined scents would have a counterproductive effect when attempting to reduce the patient's stress. Therefore this embodiment is advantageous in that it further reduces a patient's stress level.

A further preferred embodiment of the system according to the invention comprises a user interface for selecting the scent. This embodiment enables preventing from a scent that is disliked by the patient which would cause additional stress. Therefore this embodiment advantageously prevents from inducing stress with the patient rather than reducing his or her stress level.

A further preferred embodiment of the system according to the invention comprises a head set, configured for being worn by the patient during the performance of the medical procedure, incorporating the primary scent supply mechanism. In this embodiment the primary scent supply system is in close proximity of a patient's nasal organ during the whole medical procedure. This results in more effective scent delivery. More effective scent delivery allows for reducing the size of consecutive batches of scent, for increasing the period of time between consecutive batches of scent or for a combination thereof. Consequently this embodiment effectively circumvents both a surplus and a lack of scent. This embodiment thus has the advantage of further reducing a patient's stress level.

In a further preferred embodiment of the system according to the invention, the head set furthermore comprises a scent extraction mechanism configured for extracting scent, and wherein the controller is configured for controlling the extracting of scent according to an extraction time sequence. This embodiment is effectively capable of removing the scent not delivered to the patient's nasal organ. As a result this embodiment prevents from a surplus of scent in the room for performing the medical procedure. Consequently, this embodiment is advantageous in that it further decreases a patient's stress level.

In a further preferred embodiment of the system according to the invention, the head set incorporates the primary scent supply mechanism and the scent extraction mechanism such that these are on mutually opposite sides of a patient's nasal organ when the head set is being worn by the patient. This embodiment forces the scent to flow along the patient's nasal organ. This further increases efficiency of scent supply enabling a further reduction of the size of consecutive batches of scent, a further increase of the period of time between consecutive batches of scent or a combination thereof. Consequently this embodiment effectively circumvents both a surplus and a lack of scent. This embodiment therefore is advantageously capable of further reducing a patient's stress level.

A further preferred embodiment of the system according to the invention comprises a table top, configured for being mounted at a patient table near a patient's nasal organ during the performance of the medical procedure, incorporating the primary scent supply mechanism. In this embodiment the primary scent supply system is in close proximity of a patient's nasal organ during the whole medical procedure. This results in more effective scent delivery. More effective scent delivery allows for reducing the size of consecutive batches of scent, for increasing the period of time between consecutive batches of scent or for a combination thereof. Consequently this embodiment effectively circumvents both a surplus and a lack of scent. This embodiment hence is advantageous in that it is capable of further reducing a patient's stress level.

In a further preferred embodiment of the system according to the invention, the table top furthermore comprises a scent extraction mechanism configured for extracting scent, and wherein the controller is configured for controlling the extracting of scent according to an extraction time sequence. This embodiment is effectively capable of removing the scent not delivered to the patient's nasal organ. As a result this embodiment prevents from a surplus of scent in the room for performing the medical procedure. Consequently, this embodiment has the advantage of further decreasing a patient's stress level.

In a further preferred embodiment of the system according to the invention, the table top incorporates the primary scent supply mechanism and the scent extraction mechanism such that these are on mutually opposite sides of a patient's nasal organ when the head set is being worn by the patient. This embodiment forces the scent to flow along the patient's nasal organ. This forced flow of scent further increases efficiency of scent supply enabling a further reduction of the size of consecutive batches of scent, a further increase of the period of time between consecutive batches of scent or a combination thereof Consequently this embodiment effectively circumvents both a surplus and a lack of scent.

This embodiment therefore is advantageously capable of further reducing a patient's stress level.

A further preferred embodiment of the system according to the invention furthermore comprises a secondary scent supply mechanism, remote from the primary scent supply mechanism, configured for providing a patient with a batch of scent, and wherein the controller is arranged for controlling the secondary scent supply mechanism such that it provides during operation the batch of scent according to a secondary time sequence. This embodiment enables tailoring the supply of scent towards the actual location of the patient in the room for performing the medical procedure. That is, in dependence of the patient's location, the first scent supply mechanism, the second scent supply mechanism or both mechanisms may provide scent. Such position dependent scent supply results in more effective scent delivery which reduces the amount of scent needed to provide a calming effect. This embodiment hence is advantageous in that it is capable of further reducing a patient's stress level.

In a further preferred embodiment of the system according to the invention, the controller is configured for initiating the secondary time sequence upon detecting the patient has entered the room for performing a medical procedure. This embodiment enables providing the patient with scent precisely upon entry in the room for performing the medical procedure. This embodiment therefore prevents from a surplus as well as a lack of scent. Therefore this embodiment has the advantage of further reducing a patient's stress level.

### BRIEF DESCRIPTION OF THE DRAWINGS

FIG. 1 schematically depicts a plan view of the system according to the invention.

FIG. 2 schematically displays a primary time sequence according which consecutive batches of scent are provided during operation by the primary scent supply mechanism.

FIG. 3 schematically shows a primary time sequence having a variable period of time between consecutive batches of scent.

FIG. 4 schematically displays a primary time sequence wherein consecutive pulses of scent are provided with mutually different sizes.

FIG. 5 schematically shows an embodiment comprising a head set incorporating the primary supply mechanism.
FIG. 6 schematically shows a primary time sequence and an extraction time sequence.

FIG. 7 schematically depicts an embodiment comprising a table top incorporating the primary supply mechanism.

FIG. 8 schematically depicts a plan view of an embodiment of the system according to the invention comprising a secondary scent supply mechanism.

FIG. 9 schematically depicts a primary and a secondary time sequence.

### DETAILED DESCRIPTION OF THE EMBODIMENTS

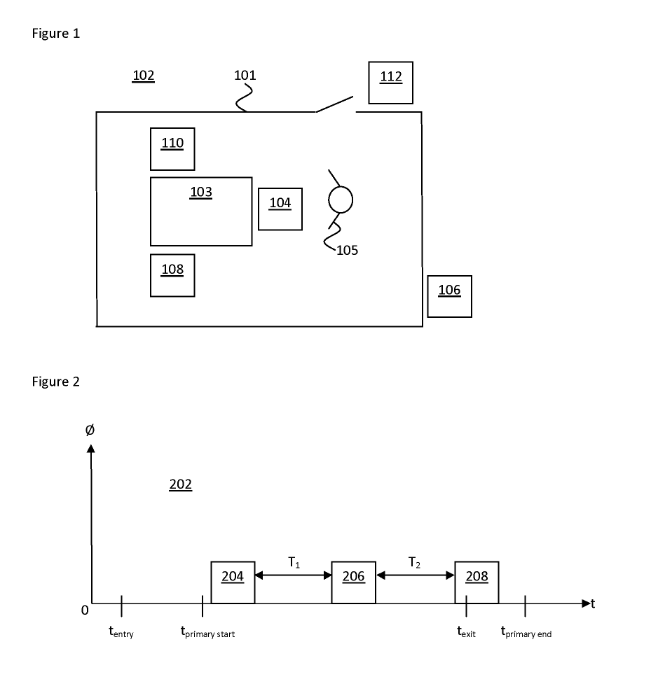

FIG. 1 depicts a plan view of a system (102) for generating an ambient healing environment in room (101) and more particularly a room for performing a medical procedure. The system (102) comprises a primary scent supply mechanism (104) known per se to the skilled person and configured for providing a patient (105) with consecutive batches of scent, and a controller (106) known per se to the person skilled in the art and arranged for controlling the primary scent supply mechanism such that it provides during operation the consecutive batches of scent intermittently according to a primary time sequence. Preferably the controller (106) is connected wirelessly to the primary scent supply mechanism (104). The controller (106) may but need not be situated in the room (101). In the room (101) an imaging device (103) is situated for performing the medical procedure. Preferably the primary scent supply mechanism (104) is situated near said imaging device (103), i.e. in close proximity of the patient, since patient stress is expected to peak when the patient (105) undergoes the medical procedure at the imaging device (103).

FIG. 2 schematically depicts a primary time sequence (202), i.e. scent mass flow rate Ø generated by the primary supply mechanism (104) as a function of time t, according which consecutive batches (204, 206, 208) are provided by the primary scent supply mechanism (104). In the specific example of FIG. 2, the consecutive batches (204, 206, 208) have identical size in terms of the amount of scent. Anyhow, as will come apparent from the embodiments discussed below, consecutive batches of scent may differ in size in response to the amount of scent in the room (101) or due to a pre-programmed pattern. In the particular example of FIG. 2, the period of time T₁ between the consecutive batches (204) and (206) equals the period of time T₂ between the consecutive batches (206) and (208). From the embodiments discussed below, it will become clear that the period of time between consecutive pulses need not be fixed but may rather fluctuate in dependence of the amount of scent in the room (101). It should be noted the primary time sequence (202) is not limited to a number of 3 consecutive batches; the primary time sequence (202) may last as long as it takes to perform the medical procedure hence may comprise numerous consecutive batches of scent.

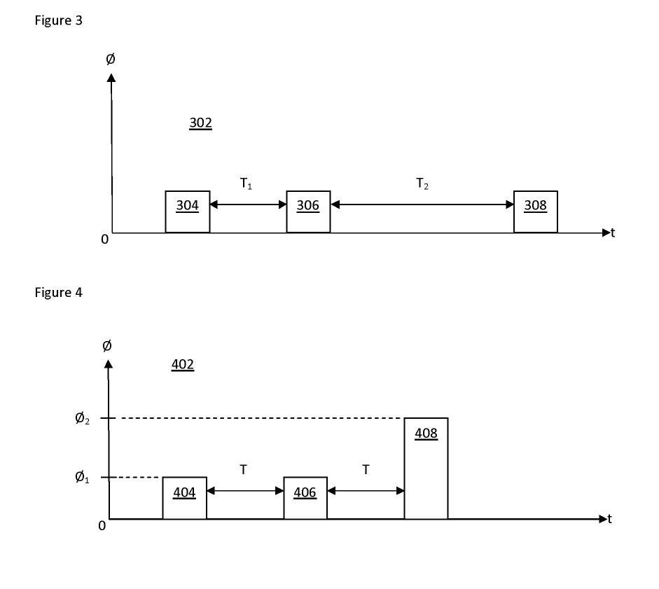

In a specific embodiment, the system (102) comprises a sensor (108) for generating a measurement signal indicative for an amount of scent in the room (101). In this particular example the controller (106) is configured for adapting—in response to the measurement signal—a period of time between consecutive batches of scent. Referring to FIG. 3 which displays a primary time sequence (302), the controller (106) increases a period of time T₁ between consecutive batches of scent (3040 and (306) to a period of time T₂ between consecutive batches of scent (306) and (308) in response to measuring a surplus of scent in the room (101). The sensor (108) may measure the scent molecules directly or use an odorless substance that is intentionally added to the liquid comprising to facilitate such measurement.

In a particular embodiment, the system (102) comprises a sensor (110) for generating a measurement signal indicative for an amount of scent in the room (101). Herein the controller (106) is configured for adapting—in response to the measurement signal—a size of the consecutive batches of scent. Referring to FIG. 4 which displays a primary time sequence (402), the controller (106) increases a mass flow rate Ø₁ of consecutive batches (404) and (406) to a mass flow rate Ø₂ for consecutive batch (408) in response to measuring a shortage of scent in the room (101).

Preferably, the controller (106) is configured for adapting both the period of time between consecutive batches and the size of consecutive batches. By doing so, the system (102) effectively postpones coming across the supply limits in terms of mass flow rate of the primary scent supply mechanism (104).

In a further embodiment the controller (106) is configured to initiate the primary supply time sequence in response to detecting the patient (105) has entered the room (101). Referring to FIG. 2, upon detection of patient entry at a point in time t_{entry} e.g. via RFID tracking, the controller will initiate the primary time sequence (202) at a point in time t_{primary start}.

In a further embodiment the controller (106) is configured to terminate the primary supply time sequence (202) in response to detecting the patient (105) has left the room (101). Referring to FIG. 2, upon detection of patient leave at a point in time tₑₓᵢₜ e.g. via RFID tracking, the controller will terminate the primary time sequence (202) at a point in time t_{primary end}.

In a further embodiment the controller (106) is configured to initiate extracting scent from the room (101) in response to detecting the patient (105) has left said room (101).

In a specific embodiment the system (102) comprises a user interface (112) for selecting the scent. The user interface (112) may be situated outside as well as inside the room (101).

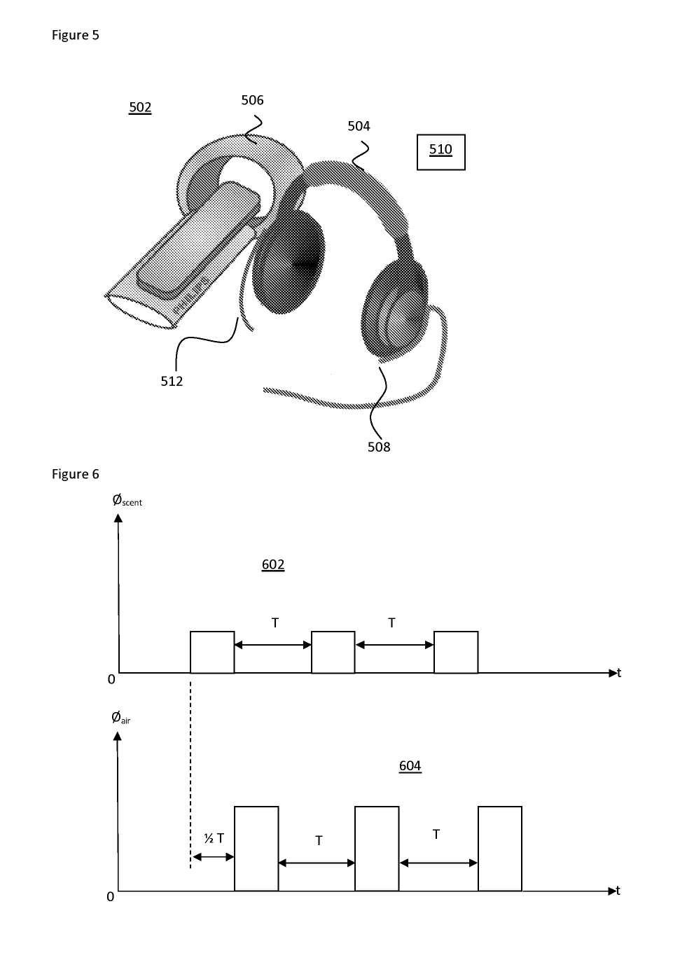

FIG. 5 schematically shows an embodiment of a system (502) for generating an ambient healing environment in a room (not shown). The system (502) comprises a head set (504) arranged for being worn by a patient (not shown) during the performance of a medical procedure. Such medical procedure is for example performed in an imaging device (506). The head set (504) incorporates a primary scent supply mechanism (508). The system (502) furthermore comprises a controller (510) for controlling the primary scent supply mechanism (508) such that it provides during operation the consecutive batches of scent intermittently according to a primary time sequence (602), i.e. scent mass flow rate Ø_{scent} as a function of time t, see FIG. 6. Referring to FIG. 5, the controller (510) is installed in the room (not shown) and has a wireless connection to the primary scent supply mechanism (508). Alternatively, the controller (510) may be incorporated in the head set (504) as well. In a specific embodiment the head set (504) comprises a scent extraction mechanism (512). Herein, the controller (510) is configured for controlling the extracting of scent according to an extraction time sequence (604), i.e. scent mass flow rate Øₐᵢᵣ as a function of time t. Referring to FIG. 6, the controller (510) will cause the scent extraction mechanism (512) to intermittently extract air according to the extraction time sequence (604). This prevents from a permanent source of noise. Preferably the extraction time sequence (604) is shifted in time half a period T relative to the primary time sequence (602) making the extraction time sequence (604) and the primary time sequence (602) mutually asynchronous. In a further specific embodiment the head set (504) incorporates the primary scent supply mechanism (508) and the scent extraction mechanism (512) such that these are on mutually opposite sides of a patient's nasal organ (not shown) when the head set (504) is being worn by the patient (not shown).

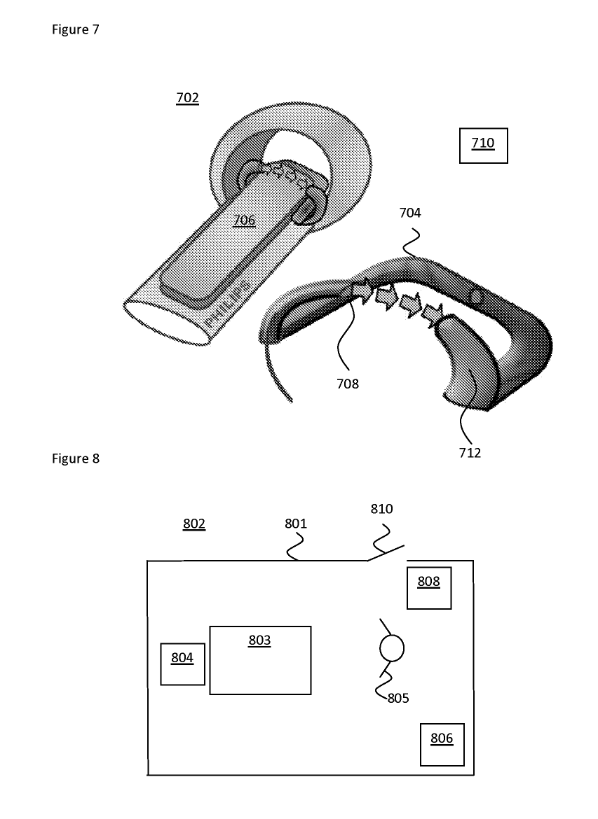

FIG. 7 schematically displays an embodiment of a system (702) for generating an ambient healing environment in a room (not shown) for performing a medical procedure. The system (702) comprises a table top (704) configured for being mounted at a patient table (706) near a patient's nasal organ (not shown) during the performance of a medical procedure. The table top (704) incorporates a primary scent supply mechanism (708). The system (702) furthermore comprises a controller (710) for controlling the primary scent supply mechanism (708) such that it provides during operation the consecutive batches of scent intermittently according to a primary time sequence (602) see FIG. 6. Referring to FIG. 7, the controller (710) is installed remote from the table top (704). The controller (710) and the table top (704) may be connected wireless. In a specific embodiment the table top (704) comprises a scent extraction mechanism (712). Herein, the controller (710) is configured for controlling the extracting of scent according to the extraction time sequence (604). In a further specific embodiment the table top (704) incorporates the primary scent supply mechanism (708) and the scent extraction mechanism (712) such that these are on mutually opposite sides of a patient's nasal organ (not shown) when the table top is mounted at the patient table (706).

FIG. 1 depicts a plan view of a room (101) for a medical procedure incorporating a system (102) for generating an ambient healing environment in said room. The system (102) comprises a primary scent supply mechanism (104) known per se to the skilled person and configured for providing a patient (105) with consecutive batches of scent, and a controller (106) known per se to the person skilled in the art and arranged for controlling the primary scent supply mechanism such that it provides during operation the consecutive batches of scent intermittently according to a primary time sequence. Preferably the controller (106) is connected wirelessly to the primary scent supply mechanism (104). The controller (106) may but need not be situated in the room (105). In the room (105) an imaging device (103) is situated for performing the medical procedure. The primary scent supply mechanism (104) preferably is situated near said imaging device (103).

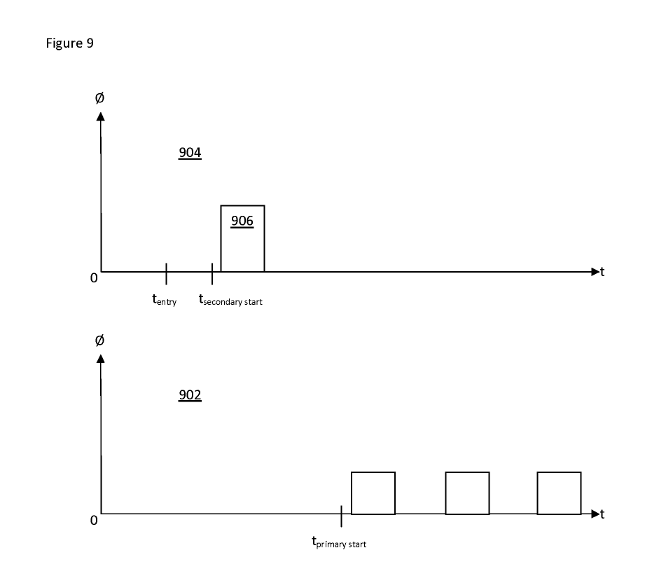

FIG. 8 depicts a plan view of a system (802) for generating an ambient healing environment in a room (801) for performing a medical procedure. The system (802) comprises a primary scent supply mechanism (804) known per se to the skilled person and configured for providing a patient (805) with consecutive batches of scent, and a controller (806) known per se to the person skilled in the art and arranged for controlling the primary scent supply mechanism such that it provides during operation the consecutive batches of scent intermittently according to a primary time sequence (902), see FIG. 9. Referring to FIG. 8, in the room (801) an imaging device (803) is situated for performing the medical procedure. In this specific example the primary scent supply mechanism (804) is situated in close proximity of said imaging device (803). The system (802) furthermore comprises a secondary scent supply mechanism (808), remote from the primary scent supply mechanism (804), configured for providing a patient (805) with a batch of scent according to a predefined secondary time sequence (904) see FIG. 9. In this specific embodiment the secondary scent supply mechanism (808) is installed in close proximity of a door (810) of the room (801). Hence, the primary and secondary scent supply mechanisms are mutually remote. In this specific embodiment the controller is (806) configured for initiating the secondary time sequence (904) upon detecting the patient (805) has entered the room (801). Referring to FIG. 9, upon detecting the patient has entered the room (801) at point in time t_{entry}, the controller (806) will initiate the primary time sequence at a point in time t_{primary start} and the secondary time sequence at a point in time t_{secondary start}. Herein, since the secondary supply mechanism (808) is close to the door (810), t_{secondary start} occurs earlier in time than t_{primary start}. More specifically, the period of time between (i) t_{secondary start} and (ii) t_{primary start}, should match the time span needed for the patient (805) to move from the door (810) to the imaging device (803). More specifically, the size of a batch of scent (906) provided by the secondary supply mechanism (8084) should be such large that this scent remains in the patient's memory until the point in time at which the primary supply mechanism (804) starts to supply scent.

While the invention has been illustrated and described in detail in the drawings and in the foregoing description, the illustrations and the description are to be considered illustrative or exemplary and not restrictive. Other combinations of embodiments are feasible. For example, the embodiment in which the system comprises a head set or the embodiment in which said system comprises a table top may be used in conjunction with the embodiments in which the system is provided with a sensor for measuring the amount of scent in the room, with a user interface and/or with patient presence detection. It is noted that the system according to the invention and all its components can be made by applying processes and materials known per se. In the set of claims and the description the word “comprising” does not exclude other elements and the indefinite article “a” or “an” does not exclude a plurality. Any reference signs in the claims should not be construed as limiting the scope. It is further noted that all possible combinations of features as defined in the set of claims are part of the invention.

1. A system for generating an ambient healing environment in a room, comprising:
a primary scent supply mechanism configured for providing a patient with consecutive batches of scent,
a controller arranged for controlling the primary scent supply mechanism such that it provides during operation the consecutive batches of scent intermittently according to a primary time sequence,
a sensor for generating a measurement signal indicative for an amount of scent in the room for performing a medical procedure,
and wherein the controller is configured for adapting in response to the measurement signal a period of time between consecutive batches of scent or
a size of the consecutive batches of scent.

2. (canceled)

3. (canceled)

4. The system according to claim 1, wherein the controller is configured to initiate the primary supply time sequence in response to detecting the patient has entered the room for performing a medical procedure.

5. The system according to claim 1, wherein the controller is configured to terminate the primary supply time sequence in response to detecting the patient has left the room for performing a medical procedure.

6. The system according to claim 1, wherein the controller is configured to initiate extracting scent from the room for performing a medical procedure in response to detecting the patient has left said room.

7. The system according to claim 1, furthermore comprising a user interface for selecting the scent.

8. The system according to claim 1, furthermore comprising a head set, configured for being worn by the patient during the performance of the medical procedure, incorporating the primary scent supply mechanism.

9. The system according to claim 7, wherein the head set furthermore comprises a scent extraction mechanism configured for extracting scent, and wherein the controller is configured for controlling the extracting of scent according to an extraction time sequence.

10. The system according to claim 7, wherein the head set incorporates the primary scent supply mechanism and the scent extraction mechanism such that these are on mutually opposite sides of a patient's nasal organ when the head set is being worn by the patient.

11. The system according to claim 1, furthermore comprising a table top, configured for being mounted at a patient table near a patient's nasal organ during the performance of the medical procedure, incorporating the primary scent supply mechanism.

12. The system according to claim 10, wherein the table top furthermore comprises a scent extraction mechanism configured for extracting scent, and wherein the controller is configured for controlling the extracting of scent according to an extraction time sequence.

13. The system according to claim 10, wherein the table top incorporates the primary scent supply mechanism and the scent extraction mechanism such that these are on mutually opposite sides of a patient's nasal organ when the head set is being worn by the patient.

14. The system according to claim 1, furthermore comprising a secondary scent supply mechanism, remote from the primary scent supply mechanism configured for providing a patient with a batch of scent, and wherein the controller is arranged for controlling the secondary scent supply mechanism such that it provides during operation the batch of scent according to a secondary time sequence.

15. The system according to claim 11, wherein the controller is configured for initiating the secondary time sequence upon detecting the patient has entered the room for performing a medical procedure.
